(19) United States
(12) Patent Application Publication
(10) Pub. No.: US 2009/0023963 A1
(43) Pub. Date: Jan. 22, 2009

(54) Noncatalytic Conversion of Carbon Tetrachloride to Perchloroethylene

(76) Inventor: Nie-Jiann Wen, Weston, MA (US)

Correspondence Address: FISH & RICHARDSON PC, P.O. BOX 1022, MINNEAPOLIS, MN, 55440-1022, US

(21) Appl. No.: 12/143,193

(22) Filed: Jun. 20, 2008

Related U.S. Application Data
(60) Provisional application No. 60/951,101, filed on Jul. 20, 2007.
(60) Provisional application No. 60/951,861, filed on Jul. 25, 2007.

(51) Int. Cl. C07C 17/26

(52) U.S. Cl. 570/189

(57) ABSTRACT

Perchloroethylene is made by thermal noncatalytic pyrolysis of carbon tetrachloride in the presence of chlorine and methyl chloride, or methane, or natural gas. Vapor carbon tetrachloride is used both as a raw material and a diluent, and reaction takes place under conditions with high consumption of maximize conversion of carbon tetrachloride to perchloroethylene and minimum formation of heavies, especially, low formation of hexchlorobenzene, another environmental undesirable chemical compound.

This patent application claims benefit from U.S. Provisional Application No. 60/951,101, filed Jul. 20, 2007, now pending, and from U.S. Provisional Application No. 60/951,861, filed Jul. 25, 2007, now pending. The complete disclosures of both applications are incorporated here by reference.

### TECHNICAL FIELD

This invention relates to a process for making perchloroethylene and hydrogen chloride by noncatalytic thermal pyrolysis of carbon tetrachloride in the presence of superheated chlorine and methyl chloride, methane, or natural gas, with superheated vapor carbon tetrachloride as both a reactant and a diluent. Under reaction conditions, carbon tetrachloride consumption is maximized and heavy ends production is minimized, especially low formation of hexachlorobenzene.

### BACKGROUND

Carbon tetrachloride is one of the halocarbons which cause destruction of the ozone layer and is therefore a relatively undesirable product. It has also been used as feedstock in producing environmentally deleterious fully halogenated chlorofluorocarbons and demand for it is therefore decreasing for this reason as well. Because of these undesirable environmental attributes of carbon tetrachloride, the demand for carbon tetrachloride is expected to have a great decrease in the coming years. On the other hand, the ecologically more benign perchloroethylene, is expected to remain in high demand because of their many practical uses, both as a solvent and as a raw material for the production of other chemicals.

Perchloroethylene can be produced by pyrolysis of carbon tetrachloride at high temperatures. As mentioned in U.S. Pat. No. 1,930,350, the suitable temperature is between 600° C. and 1500° C. As explained in U.S. Pat. No. 3,364,272, the pyrolysis process for production of perchloroethylene ordinarily requires temperatures of the order of 800° C. The pyrolysis of carbon tetrachloride discussed in U.S. Pat. No.2,447,410, requires a temperature of 1300° C. to 1400° C. High energy input to initiate and maintain the reaction, expensive materials for reactor construction, and much undesirable hexachlorobenzene and other heavy ends in the product are main drawbacks of high temperature pyrolysis process.

As discussed in U.S. Pat. No.5,315,050, and U.S. Pat. No.5,399,797, hydrogen is introduced in the reactor, with/without hydrocarbon feed. Hydrogen reacts with chlorine feed to provide heat for pyrolysis of carbon tetrachloride. But the maximum conversion of tetrachloride to perchloroethylene is only about 20%. Low conversion means large recycle of carbon tetrachloride is needed, resulting in increase in capital investment and operating cost.

Catalytic processes have also been used to produce perchloroethylene. But deactivation of catalyst and production of more unwanted heavy ends are big issues.

### SUMMARY

The present invention addresses the problems in the art by providing a process with a high conversion of carbon tetrachloride to environmental benign perchloroethylene and low formation of hexachlorobenzene.

The present invention provides a noncatalytic thermal pyrolysis process for making perchloroethylene from carbon tetrachloride, chlorine and methyl chloride, methane, or natural gas. All reactants are introduced into the reaction zone 1 after mixing. Chlorine, methyl chloride, or methane, or natural gas is introduced in amounts sufficient to provide heat for the pyrolysis of carbon tetrachloride. Carbon tetrachloride is introduced both as a reactant and a diluent in the reaction zone 1 in an amount sufficient to maintain the reaction temperature between about 500° C. and 700° C.

The amount of methyl chloride, methane or natural gas and chlorine introduced into the reaction zone depends on the carbon tetrachloride feed rate, reaction temperature, reaction pressure, residence time, etc.

Unconverted carbon tetrachloride, chlorine, perchloroethylene and hydrogen chloride is condensed in a quench tower, where perchloroethylene, hydrogen chloride and heavy ends are separated from the mixture. Unreacted carbon tetrachloride is recycled to the reactor. Compared to other processes, this process is characterized by a high conversion of carbon tetrachloride to perchloroethylene and minimum formation of heavy ends, especially low formation of hexachlorobenzene.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of the mixing scheme for introducing the feed into the reactor. After preliminary mixing, the feed is introduced into a boost jet. In the boost jet, the reactants are fully mixed and discharged into the reaction zone 1. The boost jet is inserted deeply into the reaction zone of the reactor. There are two reaction zones in the reactor.

Like reference symbols in the various drawings indicate like elements.

### DETAILED DESCRIPTION

The pyrolysis of carbon tetrachloride to form perchloroethylene is represented by the following equations:

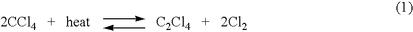

The reaction is an endothermic equilibrium reaction. High temperature and low pressure favor the formation perchloroethylene.

Since reaction 1 is endothermic, heat must be provided to maintain reaction temperature. When methyl chloride, methane, or natural gas reacts with chlorine, large amount of heat will be released. So, methyl chloride, methane, or natural gas can be used as a feed into the reactor to provide heat for pyrolysis reaction. With methyl chloride as the raw material, the total chlorination of the hydrocarbon is represented by the following equations:

CH₃Cl+3 Cl₂→CCl₄+3HCl (2)

Overall Reaction:

CH₃Cl+CCl₄+Cl₂→C₂Cl₄+3HCl (3)

With methane as a feedstock,

CH₄+4Cl₂→CCl₄+4HCl (4)

Overall Reaction:

CH₄+CCl₄+2Cl₂→C₂Cl₄+4HCl (5)

Superheated chlorine is introduced in such a rate that between 3.0% and about 10%, preferably between about 5.0% and about 7.0% volume unreacted chlorine is present in the reactor effluent. To ensure full chlorination of methyl chloride, methane or natural gas, excess chlorine is required. Excess chlorine can also prevent carbon formation. Since reaction 1 is an equilibrium reaction, too much excess chlorine will result in low conversion of carbon tetrachloride to perchloroethylene.

The carbon tetrachloride is introduced into the reaction zone to serve as both a reactant and a diluent to maintain the reaction temperature between about 500° C. and about 700° C., preferably between about 575° C. and about 625° C. Temperatures below about 500° C. result in incomplete reaction of the methyl chloride and/or methane, lower conversion of carbon tetrachloride to perchloroethylene, and formation of methylene chloride or chloroform. Higher temperatures, above about 700° C., result in carbon formation. The ratio of carbon tetrachloride to methyl chloride or methane introduced into the reactor zone will depend on methyl chloride, methane, or natural gas feedstock, the amount of chlorine introduced, and reaction conditions. Both recycled and fresh carbon tetrachloride are superheated to a higher temperature before introduced to reactor to increase carbon tetrachloride consumption.

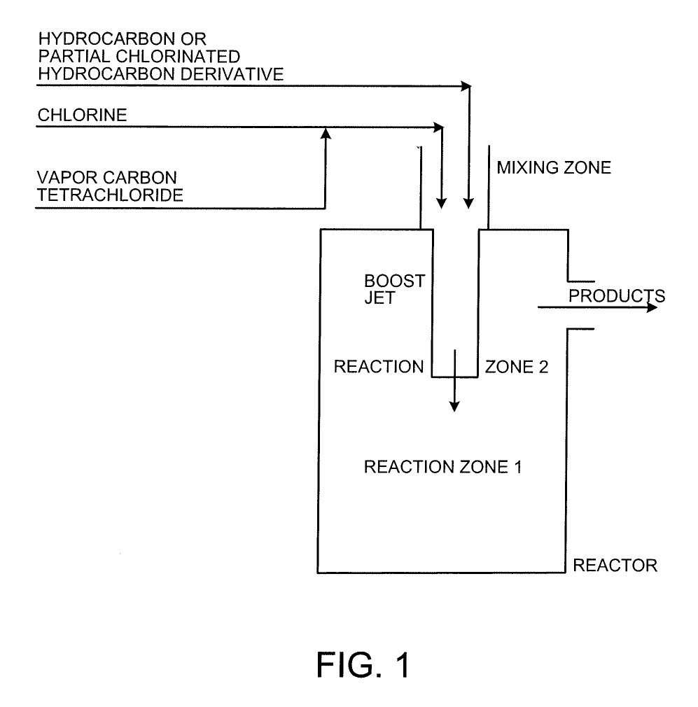

As shown in FIG. 1, the preferred method of feeding, methyl chloride, or methane, or natural gas, superheated vapor tetrachloride, and superheated chlorine are introduced into a mixing zone, then through a boost jet, which is inserted deeply into the reaction zone. The mixed feed is jetted into reaction zone 1 through the boost jet. In zone 1, both chlorination reaction and pyrolysis reaction take place. Pyrolysis reaction continues in reaction zone 2.

In order to get a good mixing, the velocity of feed mixture in the boost jet must high enough to cause vigorous turbulence and mixing in the jet. Normally, the minimum discharge velocity of the feed vapor at the end of the jet is about 30m/s, preferable velocity is between 60 m/s and 100 m/s.

Low reactor pressure favors the conversion of carbon tetrachloride to perchloroethylene. Reactor pressure can be between about 0.1 kg/cm², A and about 2 kg/cm², A. The preferred pressure is between 1.5 kg/cm²,A to 1.7 kg/cm², A.

Preferred reaction temperature is between 575° C. to 625° C.

The perchloroethylene product may be purified by quenching, condensing, and distillation.

### Example

Vaporized methyl chloride, superheated chlorine vapor (90° C.), superheated carbon tetrachloride vapor (210° C.) were introduced in a back-mixed reactor, with a volume about 14.7 m^{3.} The reactor temperature was maintained at about 595° C. and reactor pressure was about 1.7 kg/cm²,A. The reactor effluent was directly cooled in a quench tower. Quench tower overhead was condensed by several condensers. Quench tower sidedraw perchloroethylene product was further purified by distillation columns.

The results are shown below:

| Feed, kg/h | |
|---|---|
| Methyl Chloride: | 529.7 |
| Cl₂: | 1124 |
| Carbon Tetrachloride: | 3330 |
| CH₃Cl/Cl₂/CTC(molar ratio): | 1:1.51:2.06 |

| Reaction Conditions: | |
|---|---|
| Temperature, ° C.: | 595 |
| Pressure, kg/cm² G: | 0.7 |
| PCE Produced, kg/h: | 1330 |
| Fresh CTC converted to PCE, kg/h: | 854 |
| Cl₂ Excess (mole % in the reactor effluent): | 5.5 |
| CTC conversion to PCE, molar ratio: | 0.499 |
| (CTC converted to PCE/ | |
| (CTC feed + Methyl Chloride feed)) | |
| Hexachlorobenzene/PCE (kg/kg): | <0.02 |

As can be seen from the above example, by using methyl chloride and chlorine to supply reaction heat for pyrolysis, the conversion of carbon tetrachloride to perchloroethylene was high (about 50%) and net carbon tetrachloride was consumed. Low hexachlorobenzene was found in the product.

A number of embodiments of the invention have been described. Nevertheless, it will be understood that various modifications may be made without departing from the spirit and scope of the invention. Accordingly, other embodiments are within the scope of the following claims.

What is claimed is:

1. A noncatalytic thermal process for convert carbon tetrachloride to perchloroethylene, with a feedstock of methyl chloride, or methane, or natural gas, chlorine and carbon tetrachloride.

2. The process of the claim 1, wherein high conversion of carbon tetrachloride to perchloroethylene, minimum formation of heavy ends, especially, low formation of hexachlorobenzene are achieved.

3. The process of the claims 1, wherein feed and carbon tetrachloride vapor are superheated to high temperature before mixing methyl chloride or methane.

4. The process of the claims 1 or 3, wherein carbon tetrachloride is used both as a raw material and as a diluent to control the reaction temperature at about 550° C. to about 700° C.

5. The process of the claims 1 or 3, wherein the minimum velocity of reaction feed thorough the end of boost jet is about 30 m/s, and preferred velocity is between 60 m/s and 100 m/s.

6. The process of the claim 4, wherein the reaction temperature is about 575° C. to about 625° C.

7. The process of the claims 1 or 3, wherein the reaction pressure is about 0.1 kg/cm², A to 2.0 kg/cm², A, and preferred pressure is 1.5 kg/cm², A to 1.7 kg/cm², A.

8. The process of the claims 1 or 3, wherein chlorine excess in the reactor effluent is about 3.5 volume percent to 7.0 volume percent.
